(19) United States
(12) Patent Application Publication
(10) Pub. No.: US 2026/0232901 A1
(43) Pub. Date: Aug. 13, 2026

(54) SYSTEM FOR REGULATING THE CONCENTRATION OF GLUCOSE IN THE BLOOD OF A PERSON

(71) Applicant: INREDA DIABETIC B.V., Goor (NL)

(72) Inventor: Robin KOOPS, Goor (NL)

(73) Assignee: INREDA DIABETIC B.V., Goor (NL)

(21) Appl. No.: 19/156,755

(22) PCT Filed: Feb. 21, 2024
(86) PCT No.: PCT/EP2024/054382

§ 371 Date: Aug. 14, 2025

(30) Foreign Application Priority Data
Feb. 21, 2023 (NL) 2034200

(51) Int. Cl. A61M 5/168; A61M 5/14; A61M 5/142; A61M 5/172

(52) U.S. Cl. CPC A61M 5/16827; A61M 5/1407; A61M 5/142; A61M 2005/14208; A61M 2005/1726

(57) ABSTRACT

A system for regulating the concentration of glucose in the blood of a person, comprising: a device for selectively supplying at least one and preferably two substances to said person; at least one and preferably two infusion sets connectable to said device; at least one sensor for measuring a glucose concentration in the blood of said person; a controller for controlling said device such that a certain amount of a said substance is supplied per time unit, wherein said controller is arranged to determine a slope of the measured glucose concentration and if said slope exceeds a first threshold value said controller is arranged to control the device to supply a pre-injection amount of said at least one substance, said pre-injection amount being a percentage of a maximum pre-injection amount, wherein said percentage is dependent on the determined slope.

The invention relates to a system for regulating the concentration of glucose in the blood of a person, comprising:
a device for selectively supplying at least one and preferably two substances to said person,
at least one and preferably two infusion sets connectable to said device for infusing a said substance to said person;
at least one sensor for measuring a glucose concentration in the blood of said person;
a controller for controlling said device such that a certain amount of a said substance is supplied per time unit and thereby infused into the body of the person via said or a respective infusion set.

Such a system is known from for example the international patent application with number WO2007/049961 in the name of the same applicant. WO2007/049961 in particular teaches a program which incorporates an adjustable insulin injection curve represented by a first formula (ml(E)/ rise mmol/l/unit of time) and an adjustable glucagon injection curve represented by a second formula (ml(E)/ fall mmol/l/unit of time) and corresponding to an amount of insulin or glucagon respectively introduced into the body of the patient, and the nominal concentration of the glucose in the blood of the patient as a reaction thereto. Said controller for controlling said device such that a certain amount of a said substance is supplied per time unit and thereby infused into the body of the person via said or a respective infusion set may in particular be arranged to control said device to supply a certain amount that is based on said first and/or second formula. This may be referred to as the curve injection.

It is an object of the invention to improve the system described in WO2007/049961 and in particular the program thereof.

This object is achieved by providing a system according to the preamble, wherein the controller is arranged to determine a slope of the measured glucose concentration and if said slope exceeds a first threshold value said controller is arranged to control the device to supply a pre-injection amount of said at least one substance, said pre-injection amount being a percentage of a maximum pre-injection amount, wherein said percentage is dependent on the determined slope.

Such a pre-injection of said at least one substance has the purpose of preventing hypoglycemic and/or hyperglycemic glucose levels.

The slope of the measured glucose concentration is defined by the change of measured glucose concentration in time, i.e. the fall or rise of the measured glucose concentration in time. If the measured glucose concentration falls or rises above the first threshold value said pre-injection amount of said at least one substance is supplied to the person. In other words, a bolus of the at least one substance is supplied in case of rising or falling glucose values above said threshold value. This may for example be the case after the person has had a meal.

A particular advantage of such a pre-injection amount of the at least one substance may be that the person does not have to provide input into the system on the amount of carbohydrates the person is going to have during a meal, because a relatively steep rise or fall in the glucose values of the person after a meal is corrected by providing the pre-injection amount of the at least one substance.

Said program may be stored in a memory of said controller.

The controller may receive the measured glucose concentration from the sensor, wherein the sensor is able to communicate with the controller. Communication preferably takes place wirelessly, for example via a transmitter, but may alternatively take place via a wire.

It is noted that the pre-injection of the at least one substance is supplied in addition to the amount of the at least one substance that is supplied according to the programmable curve as described above. The curve injection as described above may be the regular injection of the at least one substance and the pre-injection may be supplied in addition thereto in case of a relatively steep rise or fall of the measured glucose values, i.e. a rise or fall of the measured glucose values above the first threshold value. In particular, when the slope of the measured glucose concentration drops below said threshold value, for example as a result of the pre-injection amount of substance injected into the person, the injection or supply of the at least one substance may again be based on said programable curve.

The system for regulating the concentration of glucose in the blood of a person may therefore alternatively be referred to as comprising:
a device for selectively supplying at least one and preferably two substances to said person,
at least one and preferably two infusion sets connectable to said device for infusing a said substance to said person;
at least one sensor for measuring a glucose concentration in the blood of said person;
a controller for controlling said device such that a certain amount of a said substance is supplied per time unit and thereby infused into the body of the person via said or a respective infusion set, wherein said certain amount is calculated by means of an adjustable insulin injection curve represented by a first formula (ml(E)/ rise mmol/l/unit of time) and/or an adjustable glucagon injection curve represented by a second formula (ml(E)/ fall mmol/l/unit of time) and corresponding to an amount of insulin or glucagon respectively introduced into the body of the patient, and the nominal concentration of the glucose in the blood of the patient as a reaction thereto,
characterized in that said controller is arranged to determine a slope of the measured glucose concentration and if said slope exceeds a first threshold value said controller is arranged to control the device to supply a pre-injection amount of said at least one substance, said pre-injection amount being a percentage of a maximum pre-injection amount, wherein said percentage is dependent on the determined slope, and wherein said pre-injection amount is injected in addition to/and or independent of the certain amount.

In an embodiment of the system according to the invention the maximum pre-injection amount is individually set for said person.

This allows the system to react to an individual person. The maximum pre-injection amount may for example be determined through time by monitoring how the glucose values of a particular person change after receiving the pre-injection amount of the at least one substance and may be adjusted in view thereof.

In an embodiment of the system according to the invention said device is arranged for selectively supplying two substances to said person, wherein if said slope is a positive slope exceeding a first positive threshold value said controller is arranged to control the device to supply a pre-injection amount of the first substance, and wherein if said slope is a negative slope exceeding a first negative threshold value said controller is arranged to control the device to supply a pre-injection amount of the second substance.

The pre-injection amount of the first substance, in particular insulin, is thus supplied to the person if the glucose values rise at a rate above said first positive threshold value. The pre-injection amount of the second substance, in particular glucagon, is thus supplied to the person if the glucose values fall at a rate below said first negative threshold value

In an embodiment of the system according to the invention said system is arranged such that if said slope is:
between said first positive threshold value and a second positive threshold value that is higher than the first positive threshold value between 65% and 80% of said maximum pre-injection amount of the first substance is supplied;
between said second positive threshold value and a third positive threshold value that is higher than the second positive threshold between 75 and 90% of said maximum pre-injection amount of the first substance is supplied, and
above said third positive threshold value 100% of said maximum pre-injection amount of the first substance is supplied.

In other words, how steeper the positive slope, i.e. how steeper the increase, of measured glucose values, the more first substance is supplied as a pre-injection shot or bolus.

Said percentage may be individually set for a particular person, for example by monitoring how the glucose values of said person respond to the pre-injection amount. This is for example also why said above described percentages of the maximum pre-injection may in general overlap for the various threshold values, but for a certain person this percentage will increase with increasing threshold value. For example, but not limited thereto, for an individual person the percentages may be 80%, 90%, 100%, and for another individual person the percentages may be 65%, 75%, 100%.

Said increasing percentages show that how steeper the slope the more and faster the pre-injection amount is injected. For example, above said third positive threshold value 100% of said maximum pre-injection amount of the first substance is immediately supplied, while at lower threshold values a smaller percentage of the maximum pre-injection is supplied and optionally the rest of the maximum pre-injection amount is injected later, as will be described below with respect to an embodiment of the invention.

In an embodiment of the system according to the invention said system is arranged such that if said slope is:
between said first negative threshold value and a second negative threshold value that is lower than the first negative threshold value between 65% and 80% of said maximum pre-injection amount of the second substance is supplied;
between said second negative threshold value and a third negative threshold value that is lower than the second negative threshold between 75% and 90% of said maximum pre-injection amount of the second substance is supplied, and
below said third negative threshold value 100% of said maximum pre-injection amount of the second substance is supplied.

In other words, how steeper the negative slope, i.e. how steeper the decrease, of measured glucose values, the more second substance is supplied as a pre-injection shot or bolus.

Said percentage may be individually set for a particular person, for example by monitoring how the glucose values of said person respond to the pre-injection amount. This is for example also why said above described percentages of the maximum pre-injection may in general overlap for the various threshold values, but for a certain person this percentage will increase with increasing threshold value. For example, but not limited thereto, for an individual person the percentages may be 80%, 90%, 100%, and for another individual person the percentages may be 65%, 75%, 100%.

Said increasing percentages show that how steeper the slope the more and faster the pre-injection amount is injected. For example, above said third positive threshold value 100% of said maximum pre-injection amount of the first substance is immediately supplied, while at lower threshold values a smaller percentage of the maximum pre-injection is supplied and optionally the rest of the maximum pre-injection amount is injected later, as will be described below with respect to an embodiment of the invention.

In an embodiment of the system according to the invention said system is arranged such that if said pre-injection amount is less than 100% of said maximum pre-injection amount and if said slope continues to increase or decrease for the first or second substance, respectively, after supplying the pre-injection amount, said controller is arranged to control the device to supply a second pre-injection amount of the respective substance.

Such a second pre-injection amount is thus supplied if it is detected by the controller based on the measured glucose values that the pre-injection amount of the substance is not sufficient to stop the increase or decrease of the slope, dependent on whether the first or second substance was supplied. This is thus the case if the rise or fall of the measured glucose concentration in time continues to increase in a positive or negative way, i.e. the slope becomes steeper in a positive or negative way, for insulin and glucagon, respectively.

The second pre-injection amount is chosen such that the total pre-injection amount including the pre-injection amount and the second pre-injection amount is maximally the maximum pre-injection amount. For example, if the pre-injection amount is 75% of the maximum pre-injection amount a second pre-injection amount of up to 25% of the maximum pre-injection amount may be supplied.

If the pre-injection amount and second pre-injection amount are less than the maximum pre-injection amount the system may optionally be arranged to supply a further pre-injection amount, as long as the maximum pre-injection amount is not exceeded.

Said pre-injection amount may alternatively also be referred to as the first per-injection amount.

In an embodiment of the system according to the invention said controller is arranged to control said device to inject a chosen amount of the second substance substantially together with injecting the pre-injection amount of the first substance.

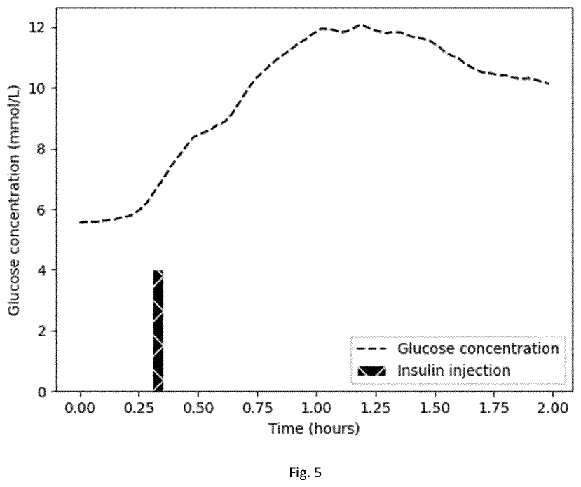

The applicant has found that if insulin, i.e. the first substance, is injected as a pre-injection amount, this insulin may be better absorbed by said person if glucagon, i.e. the second substance, is supplied before pre-injecting the first substance or at substantially the same time as pre-injecting the first substance. Practically, as will we described below with respect to FIGS. 4 and 5, it is difficult to inject the second substance before pre-injecting the first substance and such the second substance is injected substantially together with injecting the pre-injection amount of the first substance.

Substantially together may however mean together, or slightly before or after injecting the pre-injection amount of the first substance. The chosen amount of glucagon may be any suitable amount.

If said insulin pre-injection is supplied in stages of various percentages in total not exceeding the maximum pre-injection amount of insulin, said glucagon may in particular be supplied substantially together with the first pre-injection amount of insulin.

A second invention of this application relates to a system for regulating the concentration of glucose in the blood of a person, comprising:
a device for selectively supplying at least two substances to said person,
at least two infusion sets connectable to said device for infusing a respective said substance to said person;
at least one sensor for measuring a glucose concentration in the blood of said person;
a controller for controlling said device such that a certain amount of a said substance is supplied per time unit and thereby infused into the body of the person via a respective infusion set, wherein said controller is arranged to determine a slope of the measured glucose concentration and if said slope exceeds a first, positive threshold value said controller is arranged to control the device to supply a pre-injection amount of a first substance of the two substances and a certain amount of the second substance substantially together with supplying the pre-injection amount of the first substance. Independent of the particular amount of the pre-injection of the first substance, i.e. insulin, it is advantageous to supply a certain amount of the second substance, i.e. glucagon, substantially together with supplying the pre-injection amount of the first substance because of the better absorbance of insulin as described above. This second invention thus relates to the idea of supplying glucagon substantially together with supplying insulin as a pre-injection amount, independent on how the pre-injection amount of insulin is calculated or whatever amount of insulin is supplied as a pre-injection amount. Also in this second embodiment the regular injection of the first and second substances is the curve-injection and the pre-injection is provided as an additional injection if said slope exceeds said threshold value.

The certain amount of the second substance, i.e. glucagon, may be chosen as desired.

Other aspects and features of this second invention will be clear based on the description of the invention as described above and below.

Where reference is made to the first substance and the second substance, the first substance is insulin and the second substance is glucagon.

The invention will be further explained with respect to figures in which:

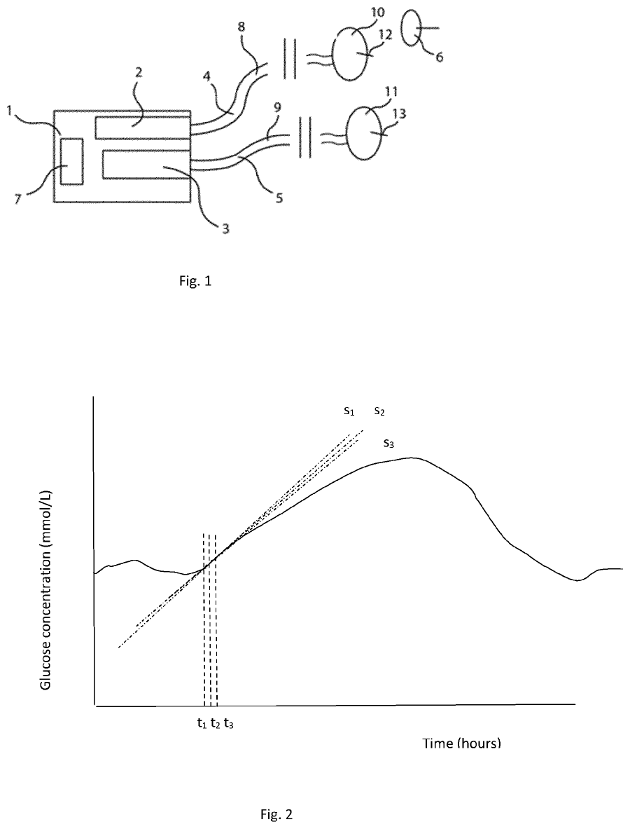

FIG. 1 schematically shows the system according to an embodiment of the invention;

FIG. 2 schematically shows a first exemplary example the glucose concentration in blood of a certain person in time;

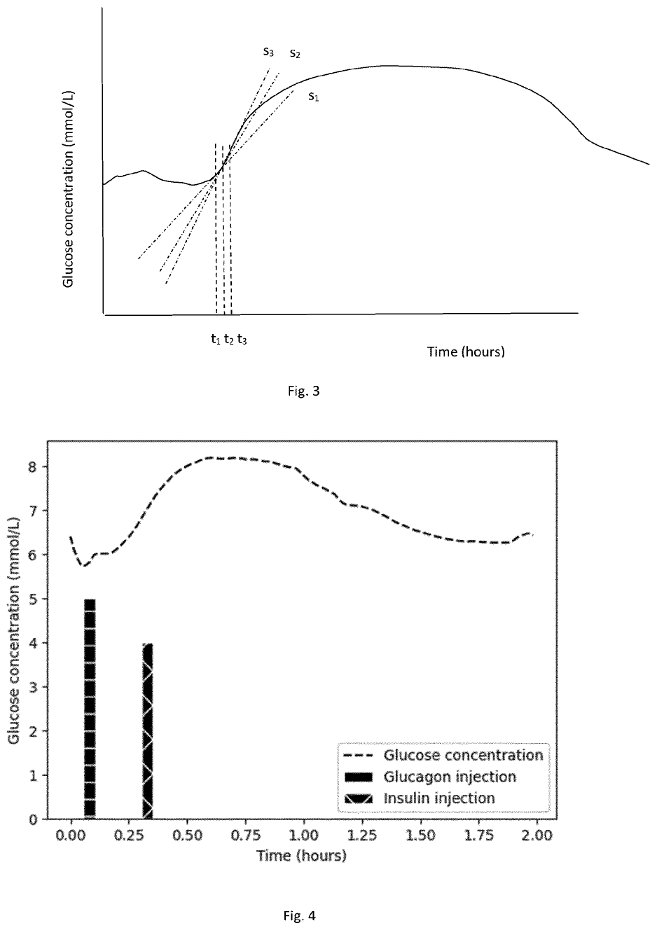

FIG. 3 schematically shows a second exemplary example the glucose concentration in blood of a certain person in time;

FIG. 4 schematically shows a third exemplary example the glucose concentration in blood of a certain person in time, and

FIG. 5 schematically shows a fourth exemplary example the glucose concentration in blood of a certain person in time.

FIG. 1 shows a system for regulating the concentration of glucose in the blood of a person. The system comprises in this exemplary embodiment a device 1 for selectively supplying in this embodiment two substances contained in respective reservoirs 2, 3, which reservoirs 2, 3 can be received in respective receiving spaces of the device 1. In this embodiment the two substances are insulin and glucagon respectively. Respective two infusion sets 4, 5 are connected to said device 1 in a fluid communicating manner with the respective reservoirs 2, 3 for infusing a said substance to said person. In this embodiment the infusion sets 4,5 each comprise a respective hose or tube 8,9 for transporting a respective substance to an infusion element 10, 11 of that infusion set 4,5. The infusion elements 10, 11 each comprise a canula 12, 13 which may be inserted into the body of the person for infusing the substance into the body of the person. In this embodiment one sensor 6 for measuring a glucose concentration in the blood of said person is provided. In this embodiment the sensor 6 is shown to be a separate sensor but alternatively the sensor may be combined with one of the infusion sets 4, 5, in particular with the infusion element 10, 11 thereof. It may be preferred to provide two glucose sensors 6, wherein optionally each sensor 6 is combined with a respective infusion set. A controller 7 is provided, in this embodiment as part of the device 1, for controlling said device 1 such that a certain amount of any one of the two substances is supplied per time unit and thereby infused into the body of the person via said or a respective infusion set 4, 5. Said certain amount may for example be chosen in accordance with the measured glucose concentration according. More in particular, said substances may be supplied according to a programmable curve, for example the adjustable glucagon injection curve represented by the first formula (ml(E)/ fall mmol/l/unit of time) or the adjustable insulin injection curve represented by the second formula (ml(E)/ rise mmol/l/unit of time) as described above. The curve injection may be the regular injection of the at least one substance. In accordance with the invention the controller 7 is arranged to determine a slope of the measured glucose concentration and if said slope exceeds a first threshold value said controller 7 is arranged to control the device 1 to supply a pre-injection amount of said at least one substance, said pre-injection amount being a percentage of a maximum pre-injection amount, wherein said percentage is dependent on the determined slope.

FIG. 2 schematically shows a first exemplary example the glucose concentration in blood of a certain person in time. This shows that until time t₁ the glucose concentration is relatively stable and within range. At time t₁ the glucose concentration suddenly starts to rise, resulting in a slope s₁ of the glucose concentration with respect to time having a relatively high value and in particular above a first threshold value. This results in said controller 7 controlling the device 1 to supply a pre-injection amount of insulin. This pre-injection amount may for example be 75% of a maximum pre-injection amount. FIG. 2 shows that as a result of the pre-injection amount of insulin the slope slightly decreases, see slope s₂ measured at time t₂ and then even further decreases, see slope s₃ at time t₃. Because of the decreasing slope, no more insulin is provided as a pre-injection but said above described curve injection may take over until the glucose values are back in range. Said sudden rise of the glucose value may for example occur when a person has had a meal and the device according to the invention is thus arranged to automatically correct for such steep rising glucose values.

FIG. 3 schematically shows a second exemplary example of the glucose concentration in the blood of a certain person in time. This shows that until time ti the glucose concentration is relatively stable and within range. At time ti the glucose concentration suddenly starts to rise, resulting in a slope s₁ of the glucose concentration with respect to time having a relatively high value and in particular above a first threshold value. This results in said controller 7 controlling the device 1 to supply a pre-injection amount of insulin. This pre-injection amount may for example be 75% of a maximum pre-injection amount. FIG. 3 shows that despite the injection of the pre-injection amount of insulin the slope continues to decrease and is even steeper, i.e. has a higher value, at time t₂, see slope s₂. In accordance with the invention the controller 7 is arranged to inject a second pre-injection amount to said person, which second pre-injection amount is for example 15% of the maximum pre-injection amount, such that in total 90% of the maximum pre-injection amount is injected. FIG. 3 shows that despite the injection of the second pre-injection amount of insulin the slope continues to decrease and is even steeper, i.e. has a higher value, at time t₃, see slope s₃. In accordance with the invention the controller 7 is arranged to inject a third pre-injection amount to said person, which second pre-injection amount is for example 10% of the maximum pre-injection amount, such that in total 100% of the maximum pre-injection amount is injected. Because in total 100% of the maximum pre-injection amount is injected, no further pre-injections amount are injected and the regular curve injection takes over. FIG. 3 shows that the three pre-injections together provide the result that the slope flattens and decreases such that after reaching a top glucose concentration in the blood after some hours, the glucose concentration starts to decrease and returns to being back in range.

The skilled person will understand that similar graphs as FIGS. 2 and 3 are applicable for glucagon, but wherein glucagon is supplied as a pre-injection in case of a sudden decrease in glucose values, i.e. a negative slope, and wherein additional pre-injections are injected if, after injecting the pre-injection.

FIGS. 4 and 5 show two other exemplary examples of the glucose concentration in the blood of a certain person in time. In the third example of FIG. 4 glucagon is injected just before the insulin pre-injection, i.e. in this example about 15 minutes before the insulin pre-injection. In the fourth example of FIG. 5 glucagon is not injected just before the insulin pre-injection. FIG. 4 shows that in case glucagon is injected just before the insulin pre-injection, the glucose concentration in the blood of the person does not rise as much as it does in the case that no glucagon is injected just before said insulin pre-injection, see FIG. 5. The applicant has found that the insulin that is supplied as the pre-injection is better absorbed by said person if glucagon is injected just before or substantially together with the insulin pre-injection, wherein as a result of the better absorption of the insulin pre-injection the glucose values do not rise as much as when no glucagon injected just before or substantially together with the insulin pre-injection. Because it is difficult to predict when an insulin pre-injection will be supplied because this depends on said slope of the measured glucose concentration which cannot be predicted but only be observed, it is difficult to supply glucagon before the insulin pre-injection. However, it is possible to supply the glucagon substantially together with the insulin pre-injection. As such, in accordance with an embodiment of the invention, glucagon may be supplied substantially together with supplying the insulin pre-injection in order to increase the absorbance of insulin by said person. Substantially together may for example mean that the glucagon injection is within 15 minutes of the insulin pre-injection, which may be maximally 15 minutes before or after of the insulin glucagon injection. Practically the glucagon injection is together with the insulin pre-injection.

It is noted that the invention is not limited to the shown embodiments but also extends to variants within the scope of the appended claims.

1. A system for regulating the concentration of glucose in the blood of a person, comprising:
a device for selectively supplying at least one substance to said person;
at least one infusion set connectable to said device for infusing a said substance to said person;
at least one sensor for measuring a glucose concentration in the blood of said person;
a controller for controlling said device such that a certain amount of a said substance is supplied per time unit and thereby infused into the body of the person via said or a respective infusion set,
wherein said controller is arranged to determine a slope of the measured glucose concentration and if said slope exceeds a first threshold value said controller is arranged to control the device to supply a pre-injection amount of said at least one substance, said pre-injection amount being a percentage of a maximum pre-injection amount, wherein said percentage is dependent on the determined slope.

2. The system according to claim 1, wherein the maximum pre-injection amount is individually set for said person.

3. The system according to claim 1, wherein said device is arranged for selectively supplying two substances to said person, wherein if said slope is a positive slope exceeding a first positive threshold value said controller is arranged to control the device to supply a pre-injection amount of the first substance, and wherein if said slope is a negative slope exceeding a first negative threshold value said controller is arranged to control the device to supply a pre-injection amount of the second substance.

4. The system according to claim 3, wherein said system is arranged such that if said slope is:
between said first positive threshold value and a second positive threshold value that is higher than the first positive threshold value between 65% and 80% of said maximum pre-injection amount of the first substance is supplied;
between said second positive threshold value and a third positive threshold value that is higher than the second positive threshold between 75 and 90% of said maximum pre-injection amount of the first substance is supplied; and
above said third positive threshold value 100% of said maximum pre-injection amount of the first substance is supplied.

5. The system according to claim 3, wherein said system is arranged such that if said slope is:
between said first negative threshold value and a second negative threshold value that is lower than the first negative threshold value between 65% and 80% of said maximum pre-injection amount of the second substance is supplied;
between said second negative threshold value and a third negative threshold value that is lower than the second negative threshold between 75% and 90% of said maximum pre-injection amount of the second substance is supplied; and
below said third negative threshold value 100% of said maximum pre-injection amount of the second substance is supplied.

6. The system according to claim 1, wherein said system is arranged such that if said pre-injection amount is less than 100% of said maximum pre-injection amount and if said slope continues to increase or decrease for the first or second substance, respectively, after supplying the pre-injection amount, said controller is arranged to control the device to supply a second pre-injection amount of the respective substance.

7. The system according to claim 1, wherein said controller is arranged to control said device to inject a chosen amount of the second substance substantially together with injecting the pre-injection amount of the first substance.

8. The system according to claim 1, claims, wherein the first substance is insulin and the second substance is glucagon.
